(19) United States
(12) Patent Application Publication
(10) Pub. No.: US 2018/0291322 A1
(43) Pub. Date: Oct. 11, 2018

(54) Multiwell Culture Devices with Perfusion and Oxygen Control

(71) Applicant: UNIVERSITY OF MIAMI, Miami, FL (US)

(72) Inventors: Ashutosh Agarwal, Miami Beach, FL (US); Siddarth Rawal, Hallandale Beach, FL (US); Giovanni Lenguito, Miami Beach, FL (US)

(21) Appl. No.: 15/766,614

(22) PCT Filed: Oct. 6, 2016
(86) PCT No.: PCT/US16/55731

§ 371 Date: Apr. 6, 2018

Related U.S. Application Data
(60) Provisional application No. 62/237,996, filed on Oct. 6, 2015.

(51) Int. Cl. C12M 3/06; B01L 3/00; C12M 1/32

(52) U.S. Cl. CPC C12M 23/16; B01L 3/502715; B01L 3/502723; B01L 2300/10; B01L 2300/0681; B01L 2300/0816; C12M 23/12

(57) ABSTRACT

A microfluidic structure is formed with one or more microfluidic channels for receiving fluid and passing the fluid through a channel in communication with a collection chamber integrated with that channel through a transition stage that allows the collection chamber to gather targets within the fluid, such as cells, including circulating tumor cells in blood. The transition stage may be formed for an asymmetrical configuration with an obstacle and chamfered face configuration. A gas permeable membrane provides perfusion control for directing gas to the collection chamber. Porous elements provide bubble venting from the fluid flow.

### CROSS-REFERENCE TO RELATED APPLICATION

This application claims the benefit of U.S. Provisional Application Ser. No. 62/237,996, filed Oct. 6, 2015, entitled “Multiwell Culture Devices with Perfusion and Oxygen Control,” which is hereby incorporated by reference in its entirety.

### STATEMENT OF GOVERNMENT SUPPORT

This invention was made with government support under 1UC4DK104208-01 awarded by the National Institutes of Health. The Government has certain rights in the invention.

### FIELD OF THE INVENTION

The present disclosure relates to multiwell fluidic devices for cell and tissue testing and, more particularly, multiwell fluidic devices having perfusion and oxygen control over cell and tissue testing.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Microfluidic platforms offer the prospect of accurately simulating the dynamic physiological conditions that cannot be achieved using standard two-dimensional plates or bio-reactors. Through design of fluidic channel architectures, controlled and efficient transport of nutrients and other soluble cues is feasible. For example, in the last decade, there has been a push to create more complex planar networks of microfluidic channels, valves, and pumps that allow for supply-control and increase the throughput by performing multiple parallel experiments using a single device. Most of the advances in the field of microfluidics allow manipulation of singular cues in the microenvironment or are amenable to simple biochemical and analytic readouts.

Unfortunately, more development is needed, in particular in regards to providing better fluid flow and perfusion control especially for platforms used for cell culturing and growth. Moreover, there remains a lack of effective mechanisms for introducing gas into microfluidic platform, although a gas like oxygen is crucial for cell growth in many instances. The lack of full control of gas introduction, as well as of perfusion control, means that for applications like diabetes, the ability to modulate culture parameters, to improve standard islet culture, and to promote efficient maturation of progenitor cells is reduced. Another problem in microfluidics is the entrainment of bubbles into the fluid path, which affects the delivery of nutrients and the collection of secrotome. There is a need for better microfluidic platforms as a result.

### SUMMARY OF THE INVENTION

The present techniques provide microplate platform structures and microfluidic channel structures. The structures provide for better fluid flow, better cell culturing, and the ability to control fluid flow and gas injection and flow. The structures offer improved islets that can be compatible with form factors such as the Society for Biomolecular Screening (SBS) form factor for filtration plates of 6, 12, 24, 96, or 384 sizes.

Described are techniques to design, build, and test structures on a microphysiological system. The present techniques further include improvements to cell culturing and methods or testing various pathologies. For example, these techniques can drive human pancreatic progenitors to create neoislets. More broadly, the different engineering controls that can be achieved with the structures described herein provide an entirely new environment to conduct mechanistic studies of human beta cell maturation, develop in vitro models of human pathologies, and test potential therapeutic strategies. Indeed, array/assay testing can now be achieved in ways not achievable before, and through, in some examples, automated perfusion control, automated collection, and automated islet operation assessment, and automated closed loop control with sensors.

In accordance with an example, an apparatus comprises: one or more microfluidic channels disposed in a filter plate, each microfluidic channel comprising an fluid inlet adjacent a proximal end and a fluid outlet adjacent a distal end and a linear channel extending between the inlet and the outlet and laying substantially in a channel plane, each microfluidic channel having a collection chamber in fluid communication with the linear channel, wherein the collection chamber extends at least partially below the channel plane and into a collection plane that is substantially parallel to the channel plane, the collection chamber having a chamfered transition stage at an inlet side of the collection chamber to introduce fluid into the collection chamber from the linear channel.

In some examples the microfluidic channels are, more generally speaking, fluidic microdevices, which can operate on microfluidic volumes or macrofluidic volumes.

In some examples, a first housing plate contains the one or more linear channels and one or more detents; and a second housing plate contains the one or more collection chambers, where the one or more detents are positioned to extend into the one or more collection chambers, and the first housing plate and the second house plate form an aligned engagement to form the one or more microfluidic channels.

In some examples, a gasket extends at least partially around a collection of the one or more microfluidic channels, the gasket being formed of a compressible material.

In some examples, a gasket extends at least partially around each of the one or more microfluidic channels, each gasket being formed of a compressible material.

In some examples, the gasket is elastomeric. In some examples, the gasket completely surrounds a collection of the one or more microfluidic channels. In some examples, the gasket completely surrounds each of the one or more microfluidic channels.

In some examples, each of the one or more microfluidic channels further contains a gas permeable membrane positioned to couple a gas into the microfluidic channel for combination within a fluid provided at the inlet.

In some examples, for each of the one or more microfluidic channels, the gas permeable membrane is positioned adjacent the fluid inlet.

In some examples, for each of the one or more microfluidic channels, the gas permeable membrane is positioned adjacent the fluid inlet to couple the gas into the microfluidic channel adjacent the proximal end.

In some examples, for each of the one or more microfluidic channels, the gas permeable membrane is positioned to provide gas directly into the collection chamber.

In some examples, a cassette is provided housing the one or more microfluidic channels and filter plate, the cassette comprises first fluid tubing connectors aligned with the fluid inlets and second fluid tubing connectors aligned with the fluid outlets.

In some examples, the cassette comprises a gas injector housings aligned with the gas permeable membranes.

In some examples, the cassette comprises a clamp positioned to sealably engage the first housing plate to the second housing plate, by compressing the gasket into sealed engagement with the first housing plate and with the second housing plate.

In some examples, an upstream portion of a microfluidic channel, i.e., upstream of a collection chamber or well, includes one or more porous elements that provide venting of any bubbles introduced in an inlet of the device without leaking fluid through the porous element, that is PTFE-based (Polytetrafluoroethylene-based).

In some examples, a first housing plate integrates a porous element bonded with ad-hoc epoxy. The element may have a water entry pressure high enough to avoid the liquid passing through the porous element, but that is still sufficiently low to allow gas to be vented. The microfluidic channel and device operate at a flow rate compatible with the amount of bubbles that are typically entrained in a microfluidic device.

In some examples, the porous element partially obstructs the liquid path, increasing the pressure drop across it, which forces the gas to go through the membrane and be vented. Meanwhile, the liquid in the device passes around the porous element trough the lumen.

In some examples, for each of the one or more microfluidic channels, the gas permeable membrane is positioned to provide gas directly below the compartments hosting the cells. A common chamber contains a gas that may be diffused through polymeric membranes that may be bonded below the devices.

### BRIEF DESCRIPTION OF THE DRAWINGS

The figures described herein depict various aspects of the system and methods disclosed herein. It should be understood that each figure depicts an example of aspects of the present systems and methods.

FIG. 1 illustrates a microfluidic channel assembly, in an example, showing each of two halves that combine to make a microfluidic channel assembly with one microfluidic channel.

FIG. 2 illustrates a microfluidic channel assembly, in another example, showing each of two halves that combine to make an assembly with multiple fluidic channels.

FIG. 3 illustrates a microfluidic channel assembly, in another example, having a three-part assembly that includes multiple fluidic channels when assembled.

FIG. 4A illustrates an integrated gasket plate as may be used in the assembly shown in FIG. 3.

FIGS. 4B-4E illustrate a removal gasket seal for use in a gasket plate, in accordance with an example.

FIG. 5 illustrates a three-part microfluidic channel assembly, in accordance with an example.

FIG. 6 illustrates a photograph of fluid flowing through a portion of a microfluidic channel.

FIG. 7A-7D illustrate different provide different views of a top (or bottom) plate of a microfluidic channel assembly.

FIGS. 7E and 7F illustrates a cross-sectional view of engaged microfluidic plates, at a certain longitudinal position.

FIG. 7G illustrates an example of an angled gasket seal as may be used in a microfluidic channel assembly.

FIGS. 8A-8E illustrate different views of a top (or bottom) plate of a microfluidic channel assembly.

FIG. 9 is a cross-sectional view of a transition stage, transitioning from the linear channels of a microfluidic channel to a collection chamber.

FIG. 10 illustrates a side view of a microfluidic structure having a fluid inlet in fluid communication with a linear channel and a fluid outlet also coupled to the linear channel. An expanded view of a collection chamber and gas permeable membrane of the structure is also shown.

FIG. 11A illustrates a microfluidic structure having fluid inlet and outlet connections as well as gas inlet and outlet connections, to allow for perfusion of gas into a collection chamber.

FIG. 11B illustrates a cross-sectional view of microfluidic structure showing a gas channel and collection chamber.

FIG. 12 illustrates an example cassette component for a microfluidic channel assembly, showing a perspective view of a fabricated chip connector and an expanded illustration of example ports used in the assembly.

FIG. 13 illustrates a schematic of gas and fluid exchange as may be achieved with microfluidic channel assemblies.

FIG. 14A illustrates a microfluidic structure having micro-fabricated gas venting windows in separate microfluidic channels. FIG. 14B illustrates a top filter plate for the gas venting structure of FIG. 14A. FIG. 14C illustrates a close-up view of a micro-fabricated gas venting window formed in a top filter plate.

FIG. 15 illustrates an example microfluidic structure having a membrane that forms a common gas chamber cap below one or more microfluidic collection chambers.

FIG. 16 is an operational diagram of a microfluidic structure with a gas (e.g., O₂) permeable membrane for directly delivery gas to collected targets (e.g., cells) in a collection chamber formed in fluid communication with a microfluidic channel.

FIG. 17 is a plot of oxygen concentration versus time for a collection chamber exposed to gas through a permeable membrane compared to a no membrane case.

FIG. 18A is a plot of glucose flux versus time for microfluidic structures in three different configurations, (i) with a naked islet with obstacle configuration on the transition stage, (ii) an encapsulated islet with obstacle configuration on the transition stage, and (iii) an encapsulated islet with a collection chamber and no obstacle.

FIG. 18B is a plot of insulin flux versus time for the same three microfluidic structure configuration plotted in FIG. 18A.

### DETAILED DESCRIPTION

A newly designed set of fabricated microstructures are provided and that may be used as microchips and/or for microsensor testing in the form of fluidic microdevices. The devices, as described, can be fabricated from techniques including laser engraving and computerized numerical control (CNC)-based micromachining. The devices may be configured into cassette structures compatible with existing testing equipment and protocols. The devices may be scaled to large arrays of fluidic microdevices for simultaneous testing and sensing of large numbers of fluidic samples. The resulting structures may be used for high-resolution optical microscopy for assessing culture viability in a collection chamber, and function in situ on each device.

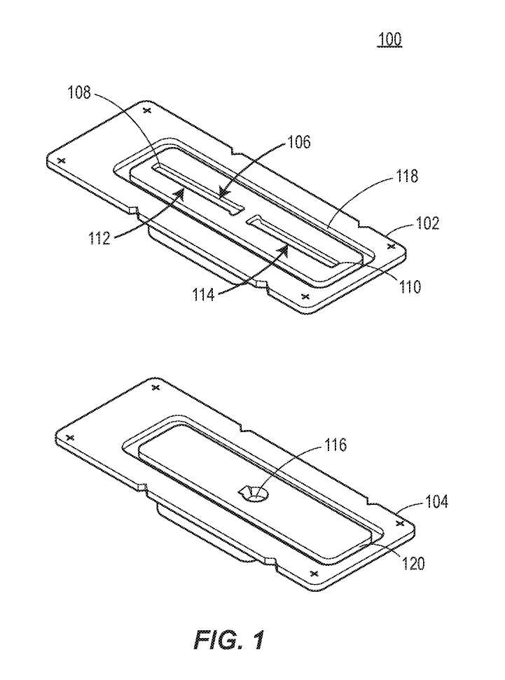

FIG. 1 illustrates a first example of a microfluidic channel assembly 100 (unassembled), formed of a first housing plate 102 (also termed a filter plate) and a second housing plate 104 (also termed a filter plate) that allow for perfusion control over a fluidic channel 106. The fluidic channel 106 is formed in the plate 102 for flow of fluid in between a proximal inlet end 108 and a distal outlet end 110. The fluidic channel is formed of a first segment 112 and a second segment 114, which are in fluid communication with each other through a collection chamber 116 that is formed in the second plate 104.

The plate 102 includes a fluid inlet at the end 108 and a fluid outlet at the end 110 that can, in some examples, receive tubing to provide for intake and expulsion of fluid, respectively.

To form a fluidic microdevice, the plates 102 and 104 are engaged to form the assembly 100. That engagement may fixedly amount the two plates 102 and 104 together or may be a releasable engagement. In some examples, the plates 102 and 104 are sandwich together, with interconnecting parts, either as part of the fluidic channel and/or collection chamber or as part of the external portions of the plates themselves. A microfluidic channel is formed of the linear channel 106 and the collection chamber 116, when the plates 102 and 104 are assembled together, one on top of the other. The plates 102, 104 may be engaged to one another from shared engagement, such as interconnecting parts. In some examples, the plates 102 and 104 are engaged through a clamp as part of a housing, cassette, or other engagement external to the fluidic channel. In some examples, the plates 102 and 104 have a tongue and groove engagement to fit the plates together and maintain sealed engagement.

In some examples, better sealing may be achieved by using a gasket seal around the microfluidic channel 106. As such, in some implementations, the plates 102, 104 each have recesses 118, 120, respectively, that can house a rigid gasket seal.

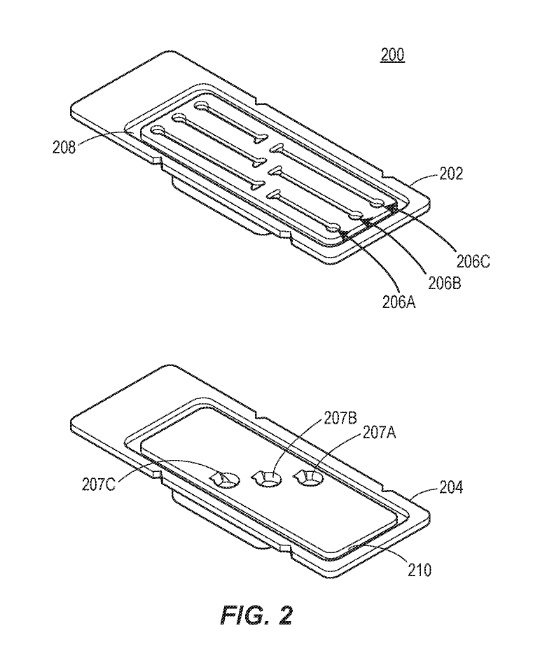

FIG. 2 illustrates another example microfluidic channel assembly 200 formed of two housing plates 202 and 204 that, when combined, form three different microfluidic channels 206A-206C. Each of the channels 206A-206C is formed of two linear channels, as shown, and a corresponding collection chamber 207A-207C coinciding with each channel. The collection chambers in the illustrated example are staggered, when examined along an axial direction of the fluidic channel, to allow for tighter placement of the channels 206A-206C.

With the staggering of collection chambers, the channels may be formed of segments of different widths. For example, each channel may be formed of two linear segments, which are of different length for channels 206A and 206C, but are of the same length for channel 206B. For the former two channels, the short linear segment may be fabricated to have a larger width than the longer linear segment. This is achieved to equalize, in some examples, the liquid volume between each of the linear segments so that they both experience the same fluid flow rates. That will establish smooth perfusion control and flow control and help increase the fluid flow through rates of the device. The ratio of the thickness between an upstream segment and a downstream segment (upstream and downstream of the collection chamber) may be chosen to achieve smooth perfusion control and fluid flow. Example ratios include 0.25×, 0.5×, 0.75×, 1.25×, 1.5×, 1.75×, 2×, 2.25×, 2.5×, and up. In some examples each segment of the linear channel can have a different depth relative to the other segment to achieve smooth perfusion control and fluid flow. Like the device 100, the plates 202 and 204 each have a recess 208 and 210, respectively, to house a sealing gasket (not shown).

The devices 100, 200, and 300 are example implementations of the techniques herein. The present techniques provide perfusion control over each fluidic channel and withdrawal of media samples from the system to allow off-chip assays of cells products (e.g., using LC/MS or other conventional analytical methodologies such as ELISA). In addition to perfusion control, the microfluidic devices can enable intimate control over oxygen microenvironments within each well. Such features may be achieved in a form factor that matches that of a multiwall culture plate to enable readouts from a commercial plate reader assays.

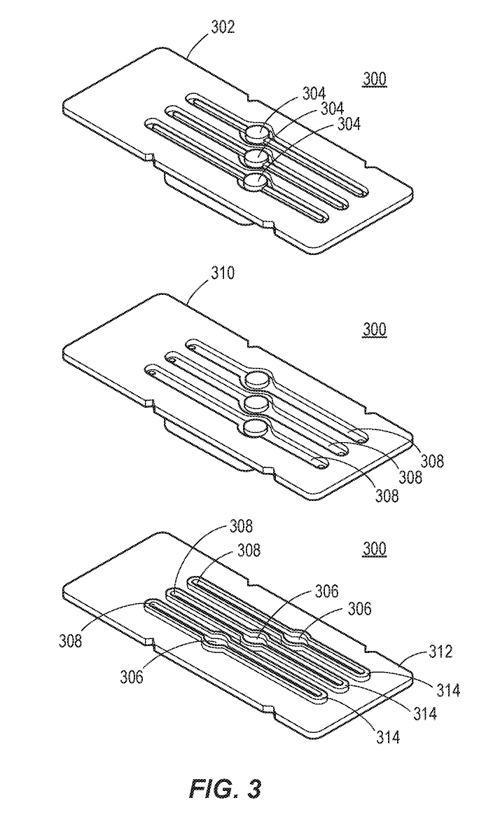

FIG. 3 illustrates a microfluidic channel assembly 300 in accordance with another example implementation, and formed of a three-plate assembly. A first plate 302 operates as a top cover plate, and may include a series of detents 304 each positioned to be aligned with a one of a series of collection chambers 306 in a series of linear channels 308, where the channels 308 and the collection chambers 306 are contained a second plate 310, serving as a bottom plate. The detents 304 function as obstacles to the fluid flow in the channels. The plates 302 and 310 may be combined together to form a microfluidic structure.

In some examples, the plate 302 (or alternatively plate 310) is configured to accept (into grooves as shown) the gasket plate 312 having a plurality of integrated rigid gasket seals 314 each aligned to seal a different one of the linear channels 308. The gasket plate 312 may be formed of two materials, a first hardened material for a platform base of the gasket plate 312 and a compressible material forming the integrated gaskets 314 that is placed on top of the base of plate 312.

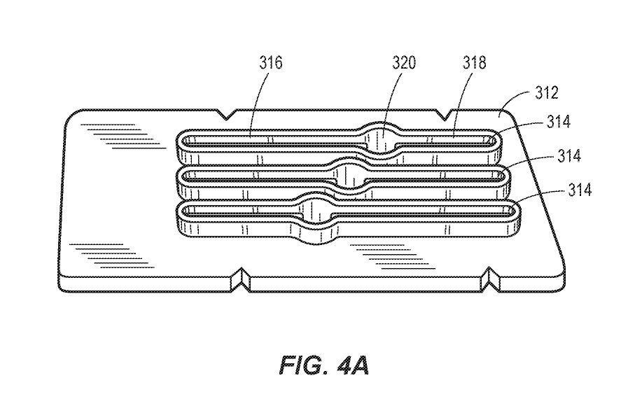

An example of the integrated gasket plate 312 is shown in FIG. 4A as formed from additive manufacturing (i.e. 3D printing) used to print the two different material types: a rigid material that works as an alignment guide, and a compressible material that is used as a gasket seal directly printed on top of the rigid material. Each gasket seal 314 has three sections, a first linear section 316 configured to provide a seal around a first portion of a microfluidic channel, a second linear section 318 configured to provide a seal around a second portion of a microfluidic channel, and a collection chamber portion 310 configured to seal around a collection chamber and to seal the inlet and outlet connection regions between the collection chamber and the microfluidic channel. FIGS. 4B-4D illustrate an example of a gasket seal 350 for use in a not-integrated gasket plate, where the gasket 350 is removable from the plate. The gasket seal 350 has linear sealing sections 352 and 354 connected to a collection chamber seal 356, in a similar manner to the gasket seal 314.

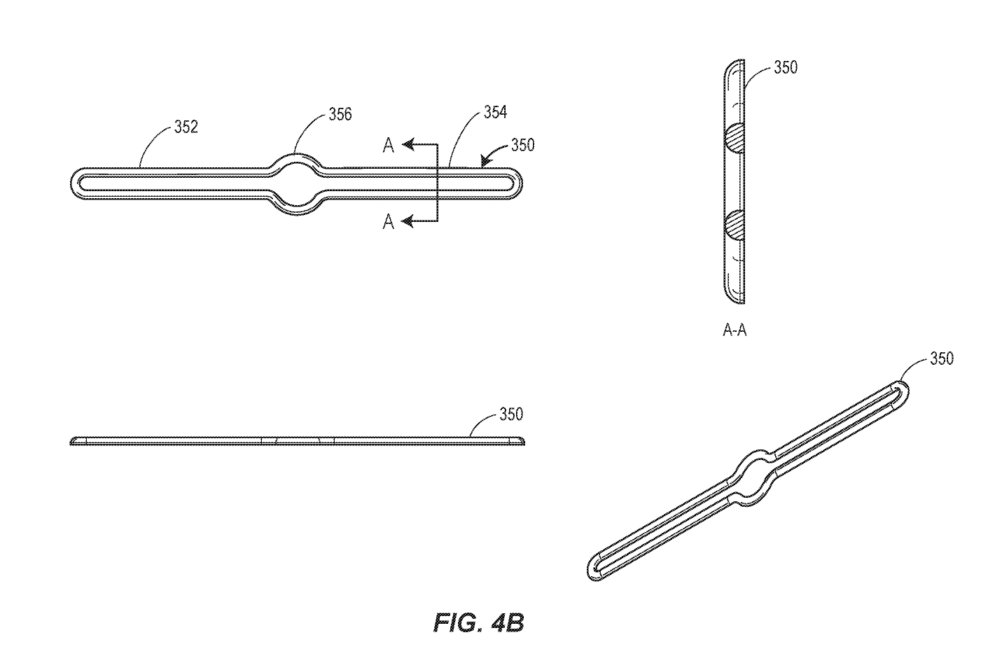

FIG. 5 provides a perspective view of a different example of microfluidic structure 300′, having a top plate 302′, a bottom plate 310′ (similar to that of FIG. 3), and showing a gasket layer 312′, in an unassembled form and realized by additive manufacturing.

In some examples, the microfluidic structures are housed in a tailored docking platform, such as a cassette having connectors for tubing and ferules, offering standardized ports. The integration of multiple devices into a single platform can offer multiple improved functions including reduced dead volume from tubing and connectors, greater ease in sterilization and maintenance of sterility, global control of oxygenation, a finer level of perfusion control, and ability to provide feedback control based on the parameters we measure using on board sensors in real time.

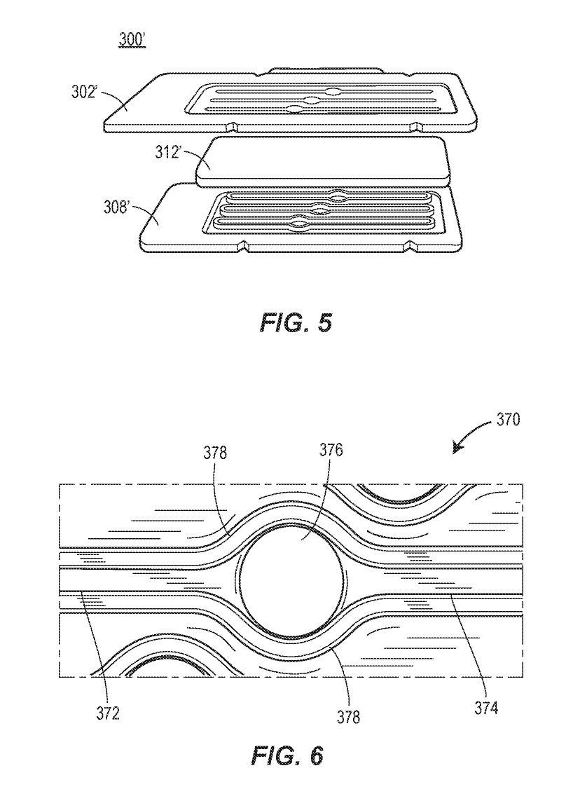

FIG. 6 illustrates a top view of fluid flowing through a portion of a microfluidic channel 370 showing linear channel segments 372 and 374, of differing widths, on either side of a collection chamber 376, with the entire structure surrounded by a compressible sealing gasket 378. The channel 370 may be one of numerous staggered channels in a planar micro-filtering structure.

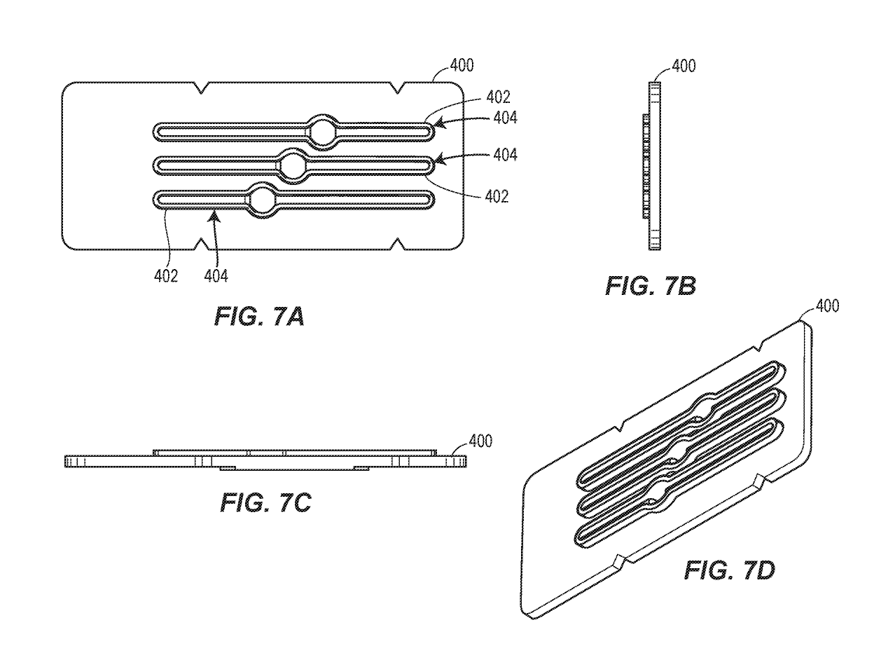

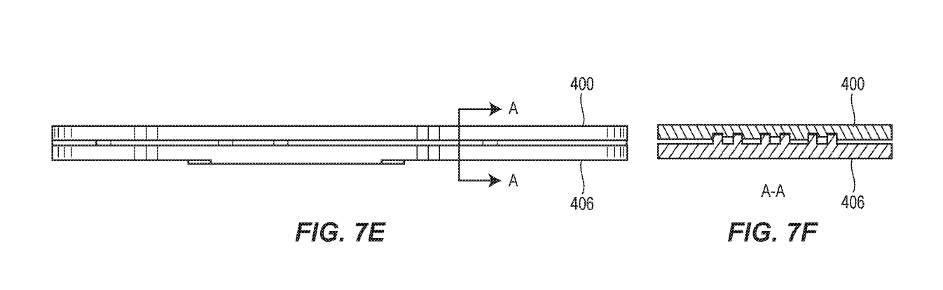

FIGS. 7A-7D illustrates engineering drawings for another example of a top plate 400, which is shown with individual rigid gaskets 402 residing inside microfluidic channels 404. FIGS. 7E and 7F illustrate a cross-sectional view of the engagement of two microfluidic structure plates 400 and 406 (see also, FIGS. 8-8E) using the integrated rigid gasket to couple the two plates. FIG. 7G illustrates an example of the angled nature of the gasket seals and grooves for each structure plate. FIGS. 8A-8E illustrate the top plate 406 corresponding to bottom plate 400.

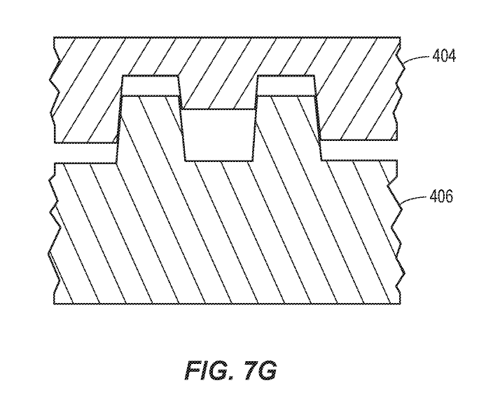

FIG. 9 illustrates an example transition stage 380 that may be used in a microfluidic device. The transition stage 380 provides a fluid transition from linear channels 382 (at an inlet side) and 384 (at an outlet side) of a microfluidic channel 386 to a collection chamber 388. In illustrated example, the collection chamber 388 includes the transition stage 380 connecting to an input side channel 382 and an output side channel 384. In the illustrated example, that transition stage 380 is asymmetric in that it has different types of transitions on the input side than on the output side. As shown, in some examples, the input side transition includes chamfered surface 390, making the transition stage a chamfered transition stage. In some examples, the chamfered surface 390 is a 45° surface; although the specific angle may be different depending on the collection chamber's geometry, and preferably the surface is angled between 30° and 60°. The chamfered surface 390 extends downward from the linear channel 382 into the collection chamber 388 and may also be, as shown, filleted in some examples. A detent 392, operating as an obstacle, above the collection chamber 388 and chamfered surface 390 may also be used as part of the transition stage 380. That detent 392 may have a right-angle inlet surface 394, a chamfered surface, or other configuration.

The shape and angle of these inlet surfaces may be determined based on the type of fluid flowing through microfluidic channel, based on the width and/or depth of the linear channels for the microfluidic channel, based on the size of the collection chamber, based on the size of the collection chamber compared to the size of the linear channels, or any other suitable design criteria. By having an asymmetric transition stage, a sufficient amount of fluid build-up may be achieved in the collection chamber, but without impeding fluid flow to the point where suitable fluid flow through the microfluidic channel cannot be achieved. This introduction of a perturbation to the fluid flow has been shown, quite counter intuitively, to benefit collection efficiency in channels such as described herein, where the dimensions on the channels are small scale dimensions. In some examples, the microfluidic devices herein may operate on small volumes of liquid, e.g., fluid volumes on the order of at or below 1000 μL, at or below 100 μL, at or below 10 μL, at or below 1 μL.

At the output end of the asymmetric transition stage 380 a right angle wall transition 396 is used to couple fluid into the output linear channel 384.

The collection chamber 388 can be disc shaped as shown in various examples described herein. That is, with a circular or semi-circular base 398. However, other shapes may be used. Further the collection chambers may have flattened bottoms, hemispherical bottoms, or otherwise. Each of the microfluidic structures described herein, sealed or unsealed, may have a transition stage as provided by example in FIG. 9.

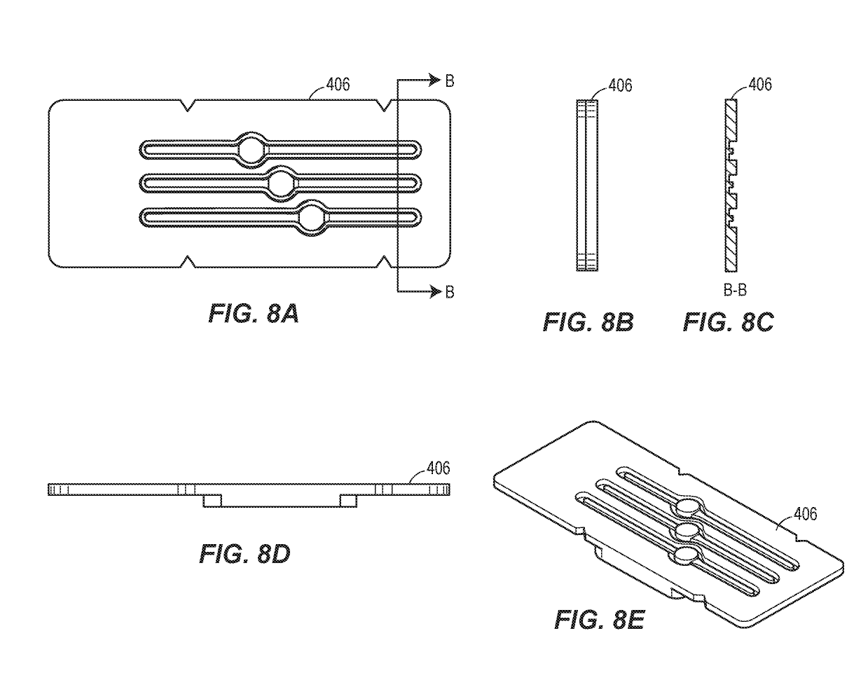

The microfluidic structures herein may include, in some examples, controllable gas and fluid inlet and outlet mechanisms. FIG. 10 illustrates a side view of a microfluidic structure 500 having a fluid inlet 502 in fluid communication with a linear channel 504. At the other end, a fluid outlet 506 is in fluid communication with the linear channel 504.

To provide a controlled introduction of gas, such as oxygen, a gas inlet 508 is provided. The inlet 508 is coupled to a thin gas channel 510 separate from the linear fluid channel 504. The gas is confined in the channel 510, which may run along the entire length of the fluid channel 504 or a portion thereof. The gas channel 510 may run parallel to the fluid channel 504 in some examples. Either way, at least in some locations, the gas channel 510 is sufficiently adjacent to the fluid channel 504 to allow for perfusion of gas into and/or out of the fluid channel 504. The channel 510, as shown in the expanded illustration, may have a gas permeable membrane 512, that polymer-based (e.g., PFA (paraformaldehyde membrane)), for exchanging gas from the channel 510 into a gas chamber 514. In this way gas may be provided directly to the site of cell growth/culturing. In other examples, a membrane between the gas channel and the fluid channel may be positioned at only points along the microfluidic channel, such as at an inlet end an outlet end or any points in between. To provide a path for gas flow, the gas channel 510 is coupled to a gas outlet 516 as a distal end of the structure 500. In some examples, the collection chamber (as shown in dashed lines) may be formed in a second plate below and engaged with the plate 500. The membrane may be any suitable polymer-based membrane, for example, with a diffusivity of oxygen through the membrane in a range of 0.1-10 m²/s.

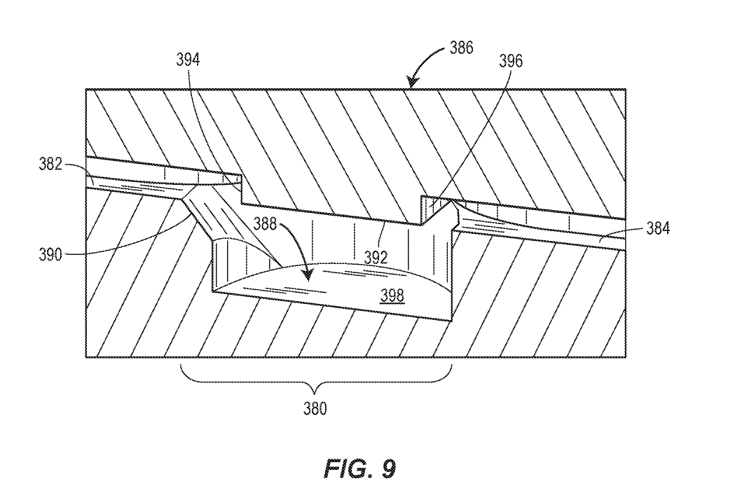

FIGS. 11A and 11B provide other views of the plate 500 showing gas and fluid ports for each of a plurality of microfluidic channels.

The microfluidic structure with gas and fluid inlet/outlet can be formed in an assembled cassette device. The cassette can provide an array of fluid connections and an array of gas connections, each individually controllable to allow for simultaneous and different microfluidic testing through a multiple microfluidic channel device.

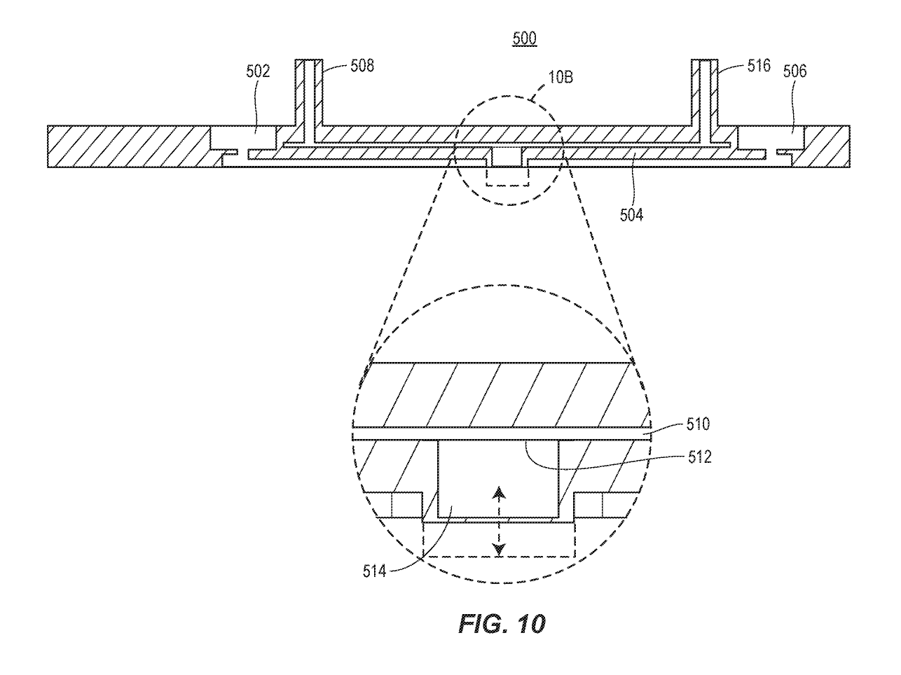

FIG. 12 illustrates a bottom portion 600 of an example cassette that may be coupled to a multiple channel microfluidic structure (not shown) used to couple to external fluid and gas inlet and outlet tubes in to a microfluidic structure, as described herein. The cassette portion 600 includes a plurality of media ports 602 that are positioned to provide fluid into fluid channels and a plurality of gas ports 604 for providing gas into a plurality of gas channels. In the expanded portion, FIG. 12 shows the fluid 602 and gas 604 inlets, while it would be understood that the other side of the cassette bottom portion 600 would have corresponding fluid and gas outlets. The cassette bottom portion 600 has a recess 606 into which a microfluidic structure (such as those described in reference to FIGS. 1-11) would be positioned in place, with the fluid and gas inlets/outlets aligned with the fluid and gas inlets of the microfluidic structure, respectively.

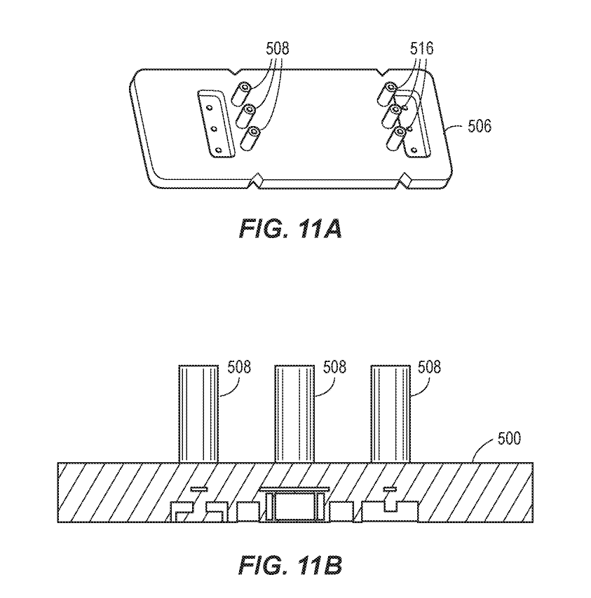

FIG. 13 illustrates a schematic of gas and fluid exchange as may be achieved with the cassette and microfluidic structure through gas perfusion. Each of the gas exchange channels represents a different collection chamber, and the exchange conditions can vary based on the properties of the gas membrane between the fluid (media) channel and the gas inlet channel. In some examples, the exchange conditions vary by introducing different gases into different gas inlet channels (not shown).

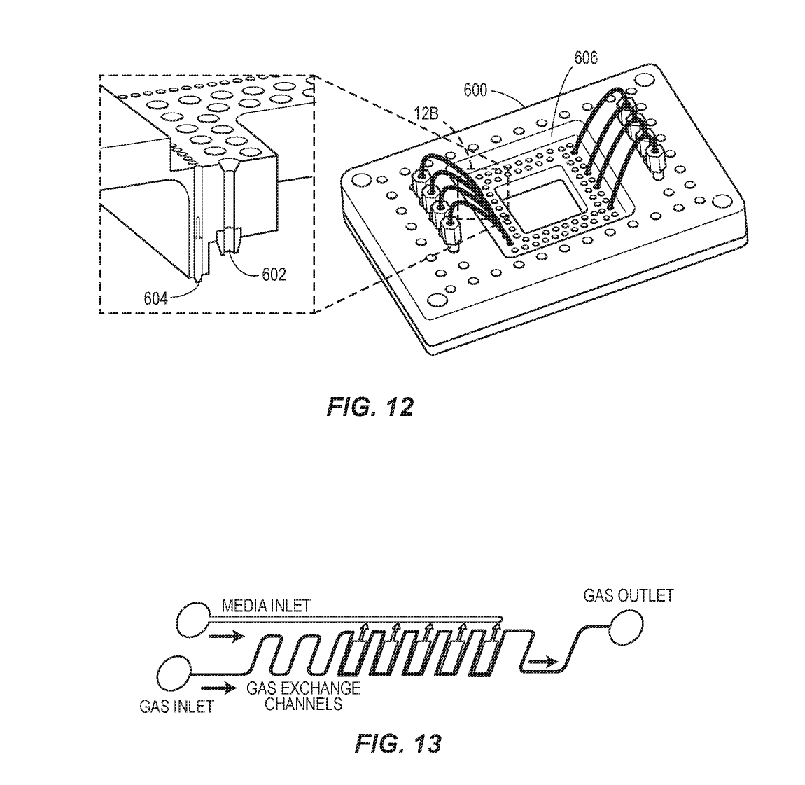

FIGS. 14A-14C illustrates a filter plate 702 (a channel plate) and a second filter plate 704 (having a plurality of a collection chambers and vents for venting the gas or bubbles away from the microfluidic channel. The venting windows 706 may be depressed openings milled into the filter plate 702. The venting windows 706 host a porous engagement element configured to capture the bubbles or gas. In some examples, a recess 712 is formed as part of the second filter plate 704 and positioned in correspondence of the venting windows 706, when the two plate device is assembled; and these venting membranes provide a gaseous release of captured gas/bubbles into the atmosphere above the structure 700.

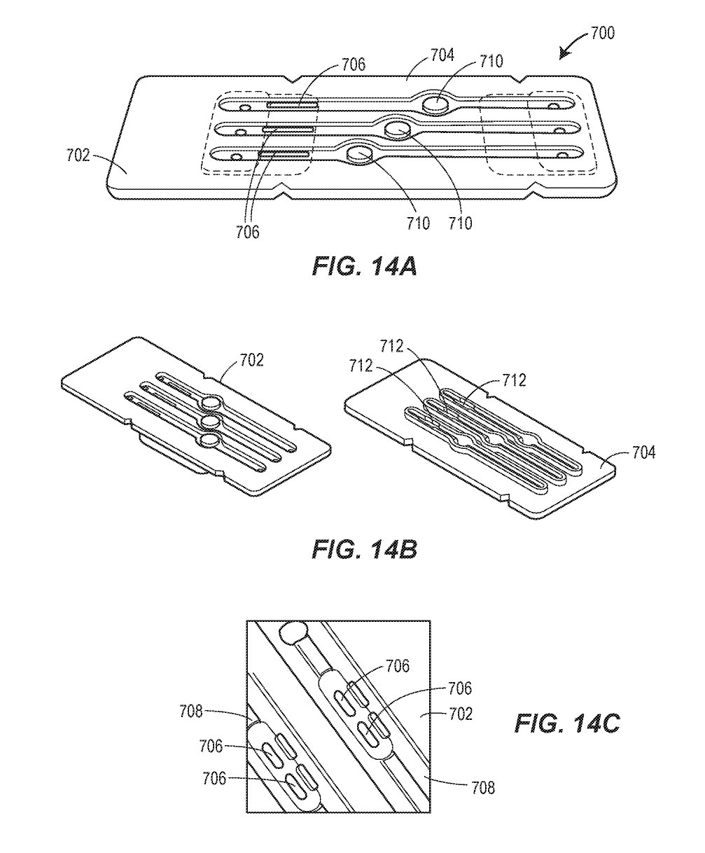

FIG. 15 illustrates another example a membrane implementation, in which filter plate 800 includes a common gas chamber cap 802 that is positioned below each of the collection chambers 804 of the filter plate 800 and provides a sealed chamber with a thin common gas chamber extending between the outer seal edge 806 of the cap 802. The common gas chamber provides the right amount of gases directly into the cell compartments through a transparent polymeric membrane 812 that may be bonded to the bottom of the filter plate 800 and extend over all three collection chambers 804. Finally the common gas chamber is sealed with a window having gas inlet 808 and gas outlet 810. The cap 802 may be bonded with ad-hoc epoxy to the bottom plate 800 and in such a way that allows for eventual small deformation of the device when loaded with clamping forces.

The structures herein can be used in any number of applications.

Oxygen Control to Preserve Islet Health and Direct Progenitor Cell Differentiation. One of the challenges in standard cell culturing is the provision of adequate oxygenation. In this respect, the “one size fits all” approach for oxygen is lacking in present systems. And this inability to sufficiently control oxygenation stands in contrast with the other physiological parameters (temperature, pH, etc.) that are controlled to exact levels. Islets are typically cultured in suspended systems at atmospheric O₂ concentration (160 mmHg). Under these conditions, islets are often exposed to sharp gradients ranging from overt hyperoxia (>100 mm Hg) to central anoxia (0 mm Hg), resulting in only a small fraction of the islet mass receiving physiological oxygenation. Considering that islets are exquisitely sensitive to both hypoxia and oxidative stress, islet death ex vivo occurs. In any event, standard culture conditions are suboptimal in delivering adequate physiological oxygenation.

Examining the correlation between oxygen delivery and islet health, we have shown dramatic gains in viability and function of pancreatic islets by careful adjustment of environmental oxygen. Of note, these gains were not observed by simply increasing oxygen tension, but via targeting the appropriate physiological oxygen range. These observations underline the importance of approximating culture conditions as closely as possible to those of the native microenvironment. Similar limitations apply to the differentiation of stem cells into islet β cells, as low differentiation efficiency indicates the need to optimize ex vivo culture to simulate their native physiological environment. Indeed, the very same oxygenation deficiencies responsible for islet death in culture may also hinder their terminal differentiation from immature progenitors. For example, oxygen acts through hypoxia inducible factor (HIF-1α; the main “oxygen sensor” of the cell) to modulate some of the key pathways involved in fate acquisition during pancreatic development, namely Wnt-β-catenin and Notch. There is a considerable influence of oxygen in regulating the balance between proliferation and differentiation in the various cellular subsets of the developing pancreas.

However, most attempts at differentiating β cells from stem cells have disregarded the “physiological niche” of pancreatic endocrine cells—a niche that conventional culture systems are ill suited to reproduce anyway. Oxygen tension modulation has a measurable effect on the differentiation of beta cells from hES cells, where tailoring oxygen to physiological conditions efficiently transitioned pancreatic progenitors (PP) into islet-like clusters.

We have used multiphysics computational analysis (COMSOL) to predict the ideal oxygen range needed to achieve physiological oxygen to the majority of the cells, defined at 70-90 mmHg; a stark contrast to the 160 mmHg typically used for cell culture. In the physiological oxygen group, the expression of most key markers of pancreatic development was significantly up-regulated versus control.

In addition to islet hormones, markers of terminal pancreatic endocrine cell maturation such as Pdx1 (28-fold), Nkx6.1 (43-fold), glucokinase (9-fold), synaptophysin (26-fold) and islet amyloid polypeptide (62-fold) were significantly (p<0.05) up-regulated. Notably, IHC for insulin and glucagon revealed superior separation of the two hormones in the oxygen-modulated group.

Thus, while standard culture conditions resulted in non-functional polyhormonal cell types, O₂ physiological targeting resulted in enhanced endocrine differentiation and increased separation of alpha and beta cells. The overall appearance and organization of the oxygen-modulated clusters was almost virtually indistinguishable from that of human isolated islets. The overall level of C-peptide release was 8-fold higher than in the control.

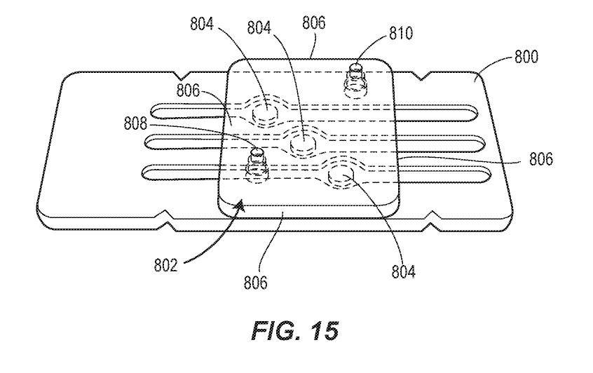

FIG. 16 illustrates a microfluidic collection with gas exchange operation of an example microfluidic structure. A fluidic inlet receives fluid. An obstacle as part of a transition stage couples the fluid into a collection chamber, with a chamfered surface and right angle combined obstruction in an asymmetrical transition stage design coupled to a fluidic outlet. A gas channel is provide for achieving gas perfusion to exchange gas directly with the cells captured in the collection chamber through a gas permeable membrane adjacent to the bottom of the collection chamber. As a result, in an example application of testing cells captured in the collection chamber, oxygen may be delivered directly into captured cells through the gas permeable membrane.

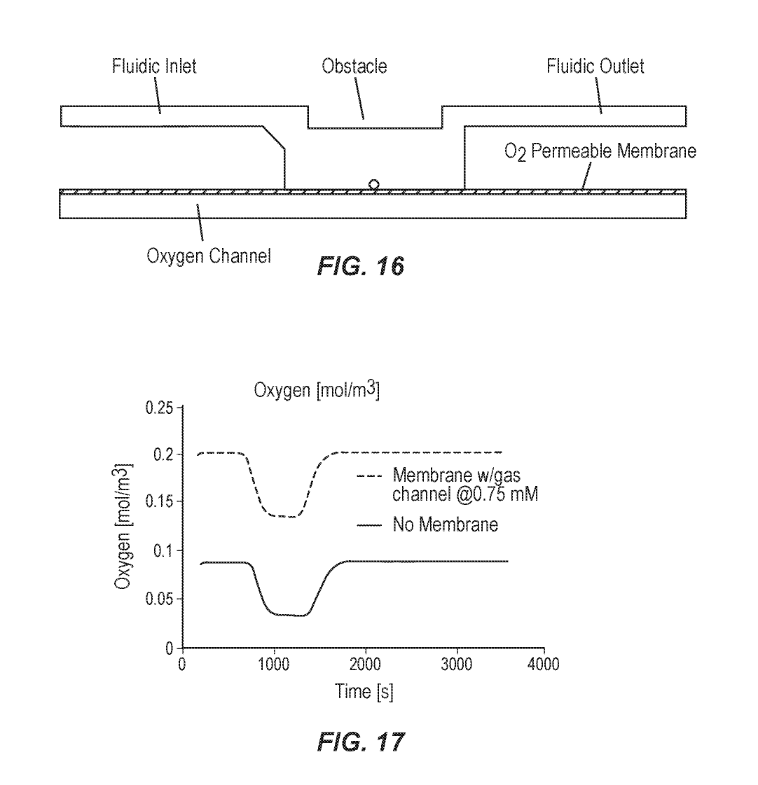

FIG. 17 shows an example of simulated oxygen control. In this specific configuration, if higher concentration of oxygen is delivered directly below the cell compartment, then it is possible to raise the oxygen concentration where cells are hosted. In this way cells do not experience a condition of hypoxia that is extremely detrimental to their functionality and survivor. Microphysiological systems provide intimate modulation of culture parameters, and incorporate critical read-outs to determine functional state

Microfluidic assemblies described herein provide a mechanism for accurately simulating the dynamic physiological conditions that cannot be achieved using standard 2D plates or bio-reactors. With the techniques described herein, and the fluidic channel architectures, control and efficient transport of nutrients and other soluble cues is now available.

The present techniques may also be implemented in organ-on-a-chip devices to allow for tissue engineering of ex vivo models of engineered biological systems. Engineering these organs present important engineering challenges that are often specific to each organ mimic, such as achieving appropriate cell type and density within the extra cellular matrix, providing perfusion media in the correct flow regime, non-invasive sensing of functional outputs, and maintaining stability and sterility over extended lifetimes of the cultures. By way of example, the present techniques may be used in organ-on-chip systems for engineering cardiac muscle, skeletal muscle, and bronchial and vascular smooth muscle.

The targeted control over oxygen microenvironments and perfusion control of the present techniques may be used for other applications, such as diabetes applications, where the ability to modulate culture parameters provides a means to improve standard islet culture and promote efficient maturation of progenitor cells. Single islets can be embedded within a fluidic well and multiple wells can be engineered within a single chip (assembly), providing a robust tool for performing multiple experiments in parallel on a single device.

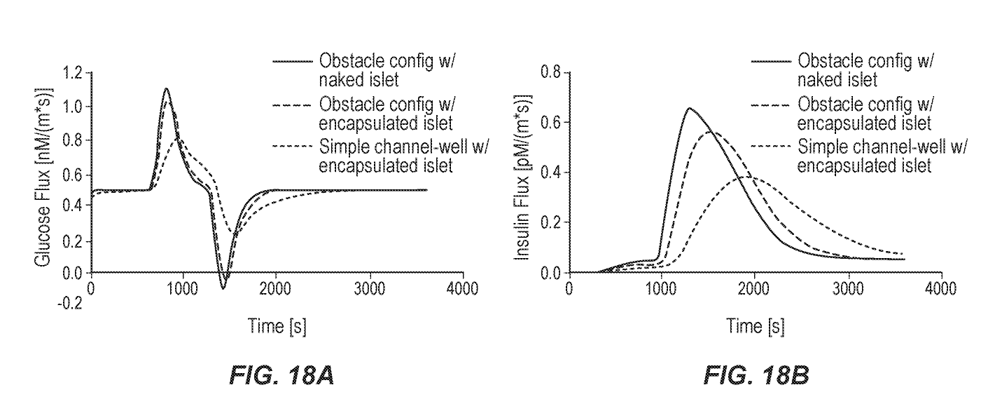

The profile of total glucose flux calculated on the boundary of the islet (FIG. 18A) shows that, compared to geometry with a simple straight microfluidic channel with no transition stage into a collection chamber, a microfluidic structure as described herein, i.e., with a divergent chamber inlet, obstacle and narrow outlet, is more effective in perfusing the islet with glucose in a timely manner. The first positive peak corresponds to the high glucose wave entering the islet, while the second negative peak corresponds to the outflux of glucose following the low glucose wave.

The profile of insulin flux at the channel outlet (FIG. 18b) demonstrates that the configuration is more effective than simple channel-well geometries. The insulin flux profile collected at the outlet shows a sharper and narrower peak that better correlates with the perfusion of high glucose. The insulin profile in the presence of an alginate capsule (100 μm thick) enveloping the islet (100 μm in diameter) is also calculated and shows a slight delay. Indeed, encapsulated islets are used for transplantation for in vivo experiments, and, therefore, are of high interest for researchers.

In another embodiment, an islet health index (“IHI”) has been developed as a result of structures described herein. The index may be generated via statistical process monitoring methodologies, which provide a statistical means to converge multiple read-outs, even temporal perfusion read-outs, into a single index score. Multivariate statistical process monitoring (SPM) methodologies may be used to provide single index scores by identifying commonalities between different read-out assessments, functional effects of variable parameters (in this case 3-D structure), and trends in time. Given that most read-outs are interrelated, multivariate techniques may be preferred (as opposed to methods such as ANOVA). The models are able to capture correlation structure between the variables and group these interdependent read-outs to parse out fewer variables that are uncorrelated to each other.

While various examples herein are described in reference to microfluidic structures, any use of microfluidic structures herein would apply to microfluidic structures as well. Therefore, the techniques described herein should be understood to apply to both microfluidic and macrofluidic domains. Fluidic microdevices as described in the foregoing may be adapted for use in either domain. As used herein, reference to microfluidic structures, microfluidic channels, etc. refers to devices having a small scale (such as a micron scale) in size and/or to devices that operate on small volume of liquid (μL, nL, pL, or fL), while macrofluidic structures, channels, etc., refers to a scale larger than microns in size and/or devices that operate a volume of liquid larger than μL.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. An apparatus comprising:
one or more microfluidic channels disposed in a filter plate, each microfluidic channel comprising a fluid inlet adjacent a proximal end and a fluid outlet adjacent a distal end and a linear channel extending between the inlet and the outlet and laying substantially in a channel plane;
each microfluidic channel having a collection chamber in fluid communication with the linear channel, wherein the collection chamber extends at least partially below the channel plane and into a collection plane that is substantially parallel to the channel plane; and
for each microfluidic channel, a chamfered transition stage positioned at an inlet side of the collection chamber and configured to introduce fluid into the collection chamber from the linear channel for collecting in the collection chamber a target in the fluid and positioned at an outlet side of the collection chamber to reintroduce the fluid into the linear channel.

2. The apparatus of claim 1, further comprising:
a first housing plate containing the one or more linear channels and one or more detents; and
a second housing plate containing the one or more collection chambers;
where the one or more detents are positioned to extend into the one or more collection chambers,
the first housing plate and the second house plate forming an aligned engagement between the first housing plate and the second housing plate to form the one or more microfluidic channels.

3. The apparatus of claim 2, wherein each collection chamber has a diameter, a spacing distance to next collection chamber, and a depth that complies with the Society for Biomolecular Screening (SBS) form factor having 6, 12, 24, 96, or 384 collection chambers.

4. The apparatus of claim 1, wherein the chamfered transition stage comprises a surface chamfered at an angle between 30° and 60°.

5. The apparatus of claim 1, further comprising a plurality of the microfluidic channels each channel having a linear channel and a collection chamber, wherein the collection chambers are each substantially disc shaped, the microfluidic channels are each substantially parallel to one another and where the collection channels are positioned in a staggered or aligned arrangement to other collection channels.

6. The apparatus of claim 1, wherein the collection chambers are disc shaped.

7. The apparatus of claim 1, wherein the collection chambers have a flat bottom or hemispherical bottom.

8. The apparatus of claims 1, wherein each transition stage comprises an asymmetric coupling with the linear channel, the asymmetric coupling having a chamfered surface at a chamber inlet of the transition stage and having a non-chamfered surface at a chamber outlet of the transition stage.

9. The apparatus of claim 8, wherein the non-chamfered transition stage is a right-angle wall transition stage.

10. The apparatus of claim 1, wherein the chamfered transition stage is filleted.

11. The apparatus of claim 1, further comprising a gasket extending at least partially around a collection of the one or more microfluidic channels, the gasket being formed of a compressible material.

12. The apparatus of claim 1, further comprising a gasket extending at least partially around each of the one or more microfluidic channels, each gasket being formed of a compressible material.

13. The apparatus of claim 12, where the gasket is elastomeric.

14. The apparatus of claim 12, wherein the gasket completely surrounds the collection of the one or more microfluidic channels.

15. The apparatus of claim 12, wherein the gasket completely surrounds each of the one or more microfluidic channels.

16. The apparatus of claim 12, further comprising:
a first housing plate containing the one or more linear channels and one or more detents; and
a second housing plate containing the one or more collection chambers,
where the one or more detents are positioned to extend into the one or more collection chambers,
the first housing plate and the second house plate forming an aligned engagement between the first housing plate and the second housing plate to form the one or more microfluidic channels,
wherein the second housing plate contains a gasket recess surrounding each of the one or more microfluidic channels, for receiving a gasket in each gasket recess.

17. The apparatus of claim 12, further comprising:
a first housing plate containing the one or more linear channels and one or more detents; and
a second housing plate containing the one or more collection chambers,
where the one or more detents are positioned to extend into the one or more collection chambers,
the first housing plate and the second house plate forming an aligned engagement between the first housing plate and the second housing plate to form the one or more microfluidic channels,
wherein the second housing plate contains a gasket recess surrounding each of the one or more microfluidic channels, for receiving an integrated gasket insert comprising a gasket for each recess.

18. The apparatus of claim 1, wherein each linear channel has first segment and a second segment separated by the collection chamber, and wherein for at least one of the linear channels one of the first segment or the second is wider than the other of the first segment or the second segment.

19. The apparatus of claim 1, wherein each linear channel has a first segment and a second segment separated by the collection chamber, and wherein for at least one of the linear channels one of the first segment or the second is longer than the other of the first segment or the second segment.

20. The apparatus of claim 1, wherein each of the one or more microfluidic channels further comprises:
a gas permeable membrane positioned to couple a gas into the microfluidic channel for combination within a fluid provided at the inlet.

21. The apparatus of claim 20, wherein for each of the one or more microfluidic channels, the gas permeable membrane is positioned adjacent the fluid inlet.

22. The apparatus of claim 20, wherein for each of the one or more microfluidic channels, the gas permeable membrane is positioned adjacent the fluid inlet to couple the gas into the microfluidic channel adjacent the proximal end.

23. The apparatus of claim 20, wherein for each of the one or more microfluidic channels, the gas permeable membrane is positioned to provide gas directly into the collection chamber.

24. The apparatus of any of claims 1-23, further comprising a cassette housing the one or more microfluidic channels and filter plate, the cassette comprising first fluid tubing connectors aligned with the fluid inlets and second fluid tubing connectors aligned with the fluid outlets.

25. The apparatus of claim 24, wherein the cassette comprising a gas injector housings aligned with the gas permeable membranes.

26. The apparatus of claim 24, wherein the cassette comprising a clamp positioned to sealable engage the first housing plate to the second housing plate, by compressing the gasket into sealed engagement with the first housing plate and with the second housing plate.

27. The apparatus of claim 1, further comprising a gas venting window disposed in the linear channel upstream of the collection chamber, the gas venting window hosing a porous element.

28. The apparatus of claim 27, further comprising:
a first housing plate containing the one or more linear channels and one or more gas venting windows ; and
a second housing plate containing the one or more collection chambers notched to accommodate porous elements in the one or more gas venting windows.

29. The apparatus of claim 1, wherein each transition stage comprises an obstacle for at least partially redirecting fluid from the linear channel to the collection chamber.
